(19) United States
(12) Patent Application Publication
(10) Pub. No.: US 2008/0305963 A1
(43) Pub. Date: Dec. 11, 2008

(54) Conductimetric biosensor device, method and system

(75) Inventors: Evangelyn C. Alocilja, East Lansing, MI (US); Zarini Muhammad-Tahir, East Lansing, MI (US)

Correspondence Address: IAN C. McLEOD, P.C., 2190 COMMONS PARKWAY, OKEMOS, MI, 48864, US

(73) Assignee: Board of Trustees of Michigan State University, East Lansing, MI (US)

(21) Appl. No.: 12/074,316

(22) Filed: Mar. 3, 2008

Related U.S. Application Data
(62) Division of application No. 10/074,499, filed on Feb. 13, 2002.

(51) Int. Cl. C40B 30/04; C12M 1/00; G01N 33/53

(52) U.S. Cl. 506/9; 435/287.2; 435/7.2

(57) ABSTRACT

A multi-array membrane strip biosensor device (10, 20) using a fluid mobile conductive polymer as reporter is described. The biosensor device (20) is designed to detect multiple analytes at low concentrations in near real-time with an electronic data collection system. The biosensor device can be small. The device can be used to detect pathogens, proteins, and other biological materials of interest in food, water, and environmental samples. The device can also be used for on-site diagnosis and against potential bioterrorism. Potential users include food processing plants, meat packaging facilities, fruit and vegetable packers, restaurants, food and water safety inspectors, food wholesalers and retailers, farms, homes, medical profession, import border crossing personnel, and the police force, military, space habitation and national security.

### GOVERNMENT RIGHTS

Not Applicable.

### BACKGROUND OF THE INVENTION

(1) Field of the Invention

The present invention relates to a biosensor device which is conductimetric, wherein conductance or resistance is measured. In particular, the present invention relates to a biosensor device which uses a fluid mobile electrically conductive polymer bound to or as a moiety of a capture reagent (such as an antibody) which captures an analyte in a fluid sample and then migrates to a capture zone where the complexed analyte is captured by another capture reagent (such as a monoclonal or polyclonal antibody) bound to (immobilized on) a substrate. The conductance or resistance is then measured. Bacteria are particularly detected by the biosensor device. Multiple detections can be accomplished simultaneously in different parallel arrays on the biosensor device.

### DESCRIPTION OF RELATED ART

Assays based upon conductivity or resistance are well known. Illustrative patents are U.S. Pat. Nos. 5,312,762 to Guiseppi-Elie and 5,670,031 to Hintsche et al. Illustrative published art is Kim et al., Biosensor & Bioelectrics 14 907, 915 (published in February of 2000). In this art, conductive polymers are used as sensors of analytes ('762 patent and Kim et al) and microsized test devices (Hirtsche et al) are used to detect an analyte. In Kim et al, a conductive polymer is bonded to conductive gold particles, which also serve as a visually detectable reagent, for a conductimetric assay. None of the prior art uses a capture reagent labeled with a conductive polymer in a sandwich type assay in the absence of conductive metal particles.

Various types of immunoassays based upon detecting a signal from a capture reagent are described in U.S. Pat. No. 5,620,845 to Gould et al.; U.S. Pat. No. 4,486,530 to David et al.; U.S. Pat. No. 5,559,041 to Kang et al.; U.S. Pat. No. 5,656,448 to Kang et al.; U.S. Pat. No. 5,728,587 to Kang et al.; U.S. Pat. No. 5,695,928 to Stewart et al.; U.S. Pat. No. 5,169,789 to Bernstein et al.; U.S. Pat. Nos. 5,177,014, 5,219,725, and 5,627,026 to O'Conner et al.; U.S. Pat. No. 5,976,896 to Kumar et al.; U.S. Pat. Nos. 4,939,096 and 4,965,187 to Tonelli; U.S. Pat. No. 5,256,372 to Brooks et al.; U.S. Pat. Nos. 5,166,078 and 5,356,785 to McMahon et al.; U.S. Pat. Nos. 5,726,010, 5,726,013, and 5,750,333 to Clark; U.S. Pat. Nos. 5,518,892, 5,753,456, and 5,620,895 to Naqui et al.; U.S. Pat. Nos. 5,700,655 and 5,985,594 to Croteau et al.; and U.S. Pat. No. 4,786,589 to Rounds et al. The aforementioned U.S. patents are hereby incorporated herein by reference.

### OBJECTS OF THE INVENTION

It is an object of the present invention to provide a device, method of use and system which enables the conductimetric (resistance or conductivity) detection of an analyte in a fluid sample. In particular, it is an object of the present invention to provide a biosensor device for use in the method and system which reliably detects the analyte by electrical conduction or resistance. Further, it is an object of the present invention to provide a device which can easily be miniaturized and which can be produced economically. These and other objects will become increasingly apparent by reference to the following description and the drawings.

### SUMMARY OF THE INVENTION

The present invention relates to a biosensor device which comprises:

a strip of a substrate having at least two zones wherein a

(1) first of the zones contains a first capture reagent bound to or as a moiety of the substrate in a defined area and spaced apart electrodes defining sides of the defined area for providing an electrical bias to the defined area; and

(2) a second of the zones containing a fluid transfer medium for supplying a fluid to the first zone, wherein the second zone comprises a second defined area containing a second capture reagent bound to or as a moiety of an electrically conductive polymer, wherein when a fluid sample containing an analyte is bound by the second capture reagent to form a complex, the complex migrates to the first zone in the medium and the analyte is bound by the first capture reagent thereby altering a conductivity or resistance of the defined area in the first zone as measured between the electrodes to detect the analyte.

Further, the present invention relates to a method for detecting an analyte in a fluid sample which comprises:

(a) providing a biosensor device which comprises:

a strip of a substrate having at least two zones wherein a

(1) first of the zones contains a first capture reagent bound to or as a moiety of the substrate in a defined area and spaced apart electrodes defining sides of the defined area for providing an electrical bias to the defined area; and

(2) a second of the zones containing a fluid transfer medium for supplying a fluid to the first zone, wherein the second zone comprises a second defined area containing a second capture reagent bound to or as a moiety of an electrically conductive polymer, wherein when a fluid sample containing an analyte is bound by the second capture reagent to form a complex, the complex migrates to the first zone in the medium and the analyte is bound by the first capture reagent thereby altering a conductivity or resistance of the defined area in the first zone as measured between the electrodes;

(b) applying the sample to the second defined area of the second zone so that when the analyte binds to the first and second capture reagents in the first zone, the conductivity or resistance of the first defined area is altered due to the presence of the conductive polymer to detect the analyte.

The present invention also relates to a system for detecting an analyte in a fluid sample which comprises:

(a) a biosensor device which comprises:

a strip of a substrate having at least two zones wherein a

(1) first of the zones contains a first capture reagent bound to or as a moiety of the substrate in a defined area and spaced apart electrodes defining the sides of the defined area for providing an electrical bias to the defined area; and

(2) a second of the zones containing a fluid transfer medium for supplying a fluid to the first zone, wherein the second zone comprises a second defined area containing a second capture reagent bound to or as a moiety of an electrically conductive polymer, wherein when a fluid sample containing an analyte is bound by the second capture reagent to form a complex, the complex migrates to the first zone in the medium and the analyte is bound by the first capture reagent thereby altering a conductivity or resistance of the defined area in the first zone as measured between the electrodes;

(b) electrical means for supplying an electrical bias between the electrodes; and

(c) measuring means for determining a change in the conductivity or resistance of the first area before and after application of the sample in the second zone to detect the analyte.

The present invention also relates to a biosensor device which comprises:

a strip of a substrate having at least two zones wherein a

(1) first of the zones contains a first antibody bound to the substrate in a defined area and spaced apart electrodes defining sides of the defined area for providing an electrical bias to the defined area; and

(2) a second of the zones containing a fluid transfer medium for supplying a fluid to the first zone, wherein the second zone comprises a second defined area containing a second antibody bound to an electrically conductive polymer, wherein when a fluid sample containing an antigen which is bound by the second antibody bound to the conductive polymer to form a complex, the complex migrates to the first zone in the medium and the antigen is bound by the first antibody thereby altering a conductivity or resistance of the defined area in the first zone as measured between the electrodes to detect the antigen.

The present invention also relates to a method for detecting an antigen in a fluid sample which comprises:

(a) providing a biosensor device which comprises:

a strip of a substrate having at least two zones wherein a

(1) first of the zones contains a first antibody bound to the substrate in a defined area and spaced apart electrodes defining sides of the defined area for providing an electrical bias to the defined area; and

(2) a second of the zones containing a fluid transfer medium for supplying a fluid to the first zone, wherein the second zone comprises a second defined area containing a second antibody bound to an electrically conductive polymer, wherein when a fluid sample containing an antigen which is bound by the second antibody bound to the conductive polymer to form a complex, the complex migrates to the first zone in the medium and the antigen is bound by the first antibody thereby altering a conductivity or resistance of the defined area in the first zone as measured between the electrodes;

(b) applying the sample to the second defined area of the second zone so that when the antigen binds to the first and second antibodies and the conductivity or resistance of the first defined area is altered due to the presence of the conductive polymer to detect the antigen.

The present invention also relates to a system for detecting an antigen in a fluid sample which comprises:

(a) a biosensor device which comprises:

a strip of a substrate having at least two zones wherein a

(1) first of the zones contains a first antibody bound to the substrate in a defined area and spaced apart electrodes on either of the sides of the defined area for providing an electrical bias to the defined area; and

(2) a second of the zones containing a fluid transfer medium for supplying a fluid to the first zone, wherein the second zone comprises a second defined area containing a second antibody bound to an electrically conductive polymer, wherein when a fluid sample containing an antigen which is bound by the second antibody bound to the conductive polymer to form a complex, the complex migrates to the first zone in the medium and the antigen is bound by the first antibody thereby altering a conductivity or resistance of the defined area in the first zone as measured between the electrodes;

(b) electrical means for supplying an electrical bias between the electrodes; and

(c) measuring means for determining a change in the conductivity or resistance of the first area before and after application of the sample in the second zone to detect the antigen.

The term “conductimetric” means that a signal is measured by means of a complex of the analyte linked directly or indirectly through a capture reagent to a conductive polymer in the biosensor device.

The term “resistance” means the electrical resistance usually measured in ohms.

The term “conductivity” means the current in amperes.

The term “analyte” means detecting a chemical or biological material including living cells in a sample which is detected by means of the biosensor device.

The phrase “capture reagent” means (1) fluid mobile reagent which selectively binds to the analyte and which can be a moiety of the conductive polymer or (2) which is bound to or a moiety of the substrate and which also selectively binds to the analyte. Included within the term “capture reagent” are selective antibodies, DNA, enzymes, proteins and chemicals which bind the analyte in the biosensor device.

The phrase “conductive polymer” means any polymer which is conductive and which is fluid mobile when bound to an analyte, particularly when bound with a capture reagent. Included within the term “conductive polymer” are polyanilines, polypyrrole, polythiophenes and which are dispersible in water and are conductive because of the presence of an anion or cation in the polymer. Other electrically conducting polymers include substituted and unsubstituted polyanilines, polyparaphenylenes, polyparaphenylene vinylenes, polythiophenes, polypyrroles, polyfurans, polyselenophenes, polyisothianapthenes, polyphenylene sulfides, polyacetylenes, polypyridyl vinylenes, biomaterials, biopolymers, conductive carbohydrates, conductive polysaccharides, combinations thereof and blends thereof with other polymers, copolymers of the monomers thereof. The polyanilines are preferred. Illustrative are the conductive polymers described in U.S. Pat. Nos. 6,333,425, 6,333,145, 6,331,356 and 6,315,926. The polymers of the present invention do not contain metals in their metallic form (i.e. Me).

The term “substrate” means a non-conductive material, such as membranes, silicon, paper, plastic or glass, which serves as a support for the biosensor.

The term “zones” means a region of the biosensor where a particular reaction or reactions occur in the biosensor device.

The term “complex” means a coupling of the capture reagent and conductive polymer with the analyte.

A “membrane” is a porous or non-porous material preferably made of nitrocellulose, fiber glass, cellulose, non-conductive biomaterials, and biopolymers, silicon, carbon nanotubes and other fluid transporting materials.

A “sandwich assay” is an assay which relies upon more than one capture reagent to selectively bind to an analyte. In the present biosensor device, one of the capture reagents is bound to or is a moiety of the substrate and the other is bound to or a moiety of the conductive polymer.

The term “multi-array” means a device for detecting multiple analytes simultaneously from the same sample.

### DESCRIPTION OF DRAWINGS

FIGS. 1A and 1B are plan views and FIG. 1C is a perspective view of a single unit or array of the conductimetric biosensor device 10 with an application membrane 11 and signal generation membrane 12 between electrodes 14A and 14B. Signal generation membrane 12 is coated on each side with the electrodes 14A and 14B made of copper and silver paste 14C and 14D, preferably 0.5 mm apart, wherein the gap 18 between electrodes 14A and 14B is the site for antibody immobilization of the analyte. The conductive polymer-labeled antibody membrane 17 is connected to the sample application membrane 11.

FIGS. 2A, 2A1, 2B, 2B1, 2C, 2C1 show plan and side views respectively of the biosensor device 10 and the sequence of detection by antibodies (Y) bound to the signal generation membrane 12 and to the polyaniline labeled antibody [symbol] After loading the sample on the application membrane 11, the solution flows up to dissolve the polyaniline-labeled antibody in 2A and 2A1 (T). The arrow indicates the direction of flow. Binding between antigen and labeled antibody takes place in 2B and 2B1. The binding complex moves to NC membrane 12, and then reacts with the immobilized antibody to generate an electrical signal in 2C and 2C1.

FIG. 3 is a plan view of a device 20 with multi-array detection. The electrodes 22A to 22E that need to be covered with silver paste. Regions or arrays 21A to 21D with width of 20 μm are sites for immobilizing different types of antigen-specific antibodies (a, b, c and d) on signal generator membrane 23.

FIG. 4 is a schematic system for testing a single multi-array device 10 or 20 including a box 30 into which the device 10 or 20 is inserted.

FIG. 5 is a plan view of the device 10 of the present invention on a wafer substrate as a non-conductive support and with a copper coating 14A and 14B connecting the electrodes 14C and 14D on membrane 12. The relative dimensions are shown.

FIG. 6 shows the various areas of the strip 10 of FIG. 5 and the function.

FIG. 6A shows the multimeter 19 electrically connect to the device 10.

FIGS. 7 to 10 are graphs showing the results of testing of water, cider and milk wherein the tests are quantitative for the number of cells detected by the device 10.

FIG. 11 is a graph showing tests of river water in Lansing, Mich. at various road intersections. The bars with diagonal slashes are for testing after 2 minutes. The bars which are black are for testing after 4 minutes. The bars which are gray with white dots are for testing after 6 minutes.

### DESCRIPTION OF PREFERRED EMBODIMENTS

The present invention particularly relates to biosensors and devices for rapid pathogen detection. The present invention provides a new class of biosensor devices particularly for the detection of pathogens such as Escherichia coli O157:H7. The conductimetric biosensor device is preferably based on antibody-antigen binding and has the sensitivity to detect as low as 100 to 10¹ cfu/mL of E. coli in 2 to 10 minutes. Detection is thus rapid, and can be automated, and computerized. Furthermore, by utilizing different antibodies the biosensor device can be adapted for the detection of different types of pathogens at the same time. The biosensor device can thus detect other disease-causing bacteria, toxins, pathogens, chemicals and potential biowarfare agents such as Salmonella and Listeria monocytogenes. The present invention can be particularly used to detect pathogens, proteins, and other biological materials of interest in food, water, and environmental samples. The biosensor device can also be used for onsite diagnostics and against potential bioterrorism. Users include food processing plants, meat packing facilities, fruit and vegetable packers, restaurants, food and water safety inspectors, food wholesalers and retailers, farms, homes, medical profession, import border crossing, the police force, military, space habitation and national security.

Particular markets for the biosensor device are, for instance, the corporate laboratories of various food processing plants and environmental agencies. Rapid, simple, and accurate on-site testing provides considerable value to food producers by ensuring high product quality, greater yields, elimination of product recalls, and reduced treatment costs. The user friendly, biological analysis system results in more effective management of food processing lines and inventories, as well as safer food and water supplies. With the biosensor device of the present invention, the consumer does not have to make a choice between speed and sensitivity.

The present invention has two principal embodiments, a first with the single unit of the biosensor and second, with the multi-array system. The preferred objective of the invention is to provide a multi-array membrane strip biosensor using conductive polymer, such as polyaniline as a reporter of a biological event using conductivity. The present invention provides:
1. A biosensor that can detect an antigen at near realtime with an electronic data collection system.
2. A conductive polymer-labeled antibody as reporter of biological event, such as antibody-antigen interaction.
3. Membrane strips in the construction of a sample application pad or membrane, capture and signal generation pad or membrane, and absorption pad or membrane that allow for the efficient transport of the sample by capillary action.
4. A multi-array system of the biosensor for multiple and simultaneous detection of different analytes (antigens, toxins, and the like) in the sample matrix.

The advantages of the present invention over previous approaches are shown in Table 1:

TABLE 1

| | Present | Previous |
|---|---|---|
| Parameters | Invention | Methods |
| Number of bacterial | Multiple | Single |
| species detected | | |
| per test | | |
| Sensitivity | 10⁰-10¹ cfu¹ | 10⁶ or 10⁷ |
| | | cfu/ml |
| Detection time | 2-10 minutes | 30-60 min or |
| after sample | | 24-48 hr |
| application | | |
| Size | 5 × 40 × 1 mm³ | bulky |
| | (strip only) | |
| | 25 × 75 × 2 | |
| | mm (total) | |
| Portability | Field-based | laboratory- |
| | | based |
| Signal measurement | Electronic | Manual or |
| | | electronic |
| Use of reagents | None | Many |
| Use of colloidal | None | Sometimes |
| gold | | |
| Skill requirement | Minimal | Training and |
| to operate | | skills required |

¹cfu—Colony forming units

### Example 1

### 1.1 Reagents

Aniline, glutaraldehyde, N,N Dimethylformamide (DMF), Tween-20, tris buffer, phosphate buffer, phosphate buffer saline (PBS) were purchased from Sigma-Aldrich (Missouri). Antibodies (Rabbit anti-E. Coli O157:H7) were obtained from Biodesign (Maine). Nitrocellulose (NC) membrane 12 with 8 μm pore size and flow rate of 160 sec per 4 cm, and cellulose membrane 13 were purchased from Millipore (Massachusetts). Fiber-glass membrane 11 grade G6 were also obtained from Millipore. Silver Kwik-stik pen for electrodes 14C and 14D was supplied from SPI (Pennsylvania). Other reagents used were of analytical grade. All chemicals and diluents were prepared with doubly deionized water with conductivity below 0.1 μS/cm.

Antibody Labeling with Polyaniline

A water-soluble polyaniline was synthesized by following a standard procedure of oxidative polymerization of aniline monomer in the presence of ammonium persulfate (Kim et al, previously discussed). A mixture of the antibody and polyaniline was left to react for 30 minutes. The conjugate was then precipitated by centrifugation (13000 rpm for 3 min) using 0.1 M Tris buffer as the blocking reagent. The conjugated antibody was diluted in 0.01 M LiCl.

### 1.2 Fabrication of Silver Electrodes

For electrical connection, a silver paste pen was used to make the electrodes 14C and 14D on the capture nitrocellulose (NC) membrane as the signal generation membrane 12. The silver paste 14C and 14D was applied liberally to ensure consistency in the flow of electricity between the copper electrodes 14A and 14B. The distance between the two silver electrodes 14C and 14D was in the capture region and was 0.5 mm wide. To increase detection sensitivity, the distance between the electrodes can be reduced to 2-20 μm by using microinterdigitated technology.

### Immobilization of Antibody on the Capture Membrane

Affinity purified antibody was directly immobilized between the two silver electrodes of the NC membrane by the following steps. First, the NC membrane was saturated in 10% (v/v) methanol for 45 minutes and left to dry. The surface of the membrane was then modified by immersing it in 0.5% (v/v) glutaraldehyde as a linking agent for 1 hour. After drying, 2.5 μl of 0.5 mg/ml of antibody was pipetted on the membrane site, and incubated at 37° C. for 1 hour. Inactivation of residual functional groups and blocking was carried out simultaneously by incubating the membrane with 0.1 M Tris Buffer, pH 7.6, containing 0.1% Tween-20 for 45 minutes. The membrane with the linked antibody was left in the air to dry before proceeding to the next step.

### Construction of One Unit of the Conductimetric Biosensor Device

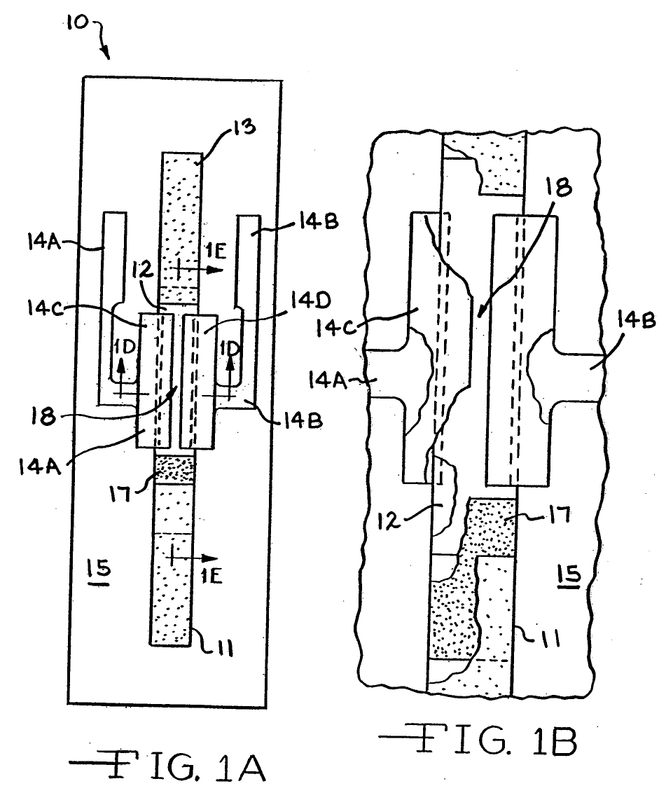

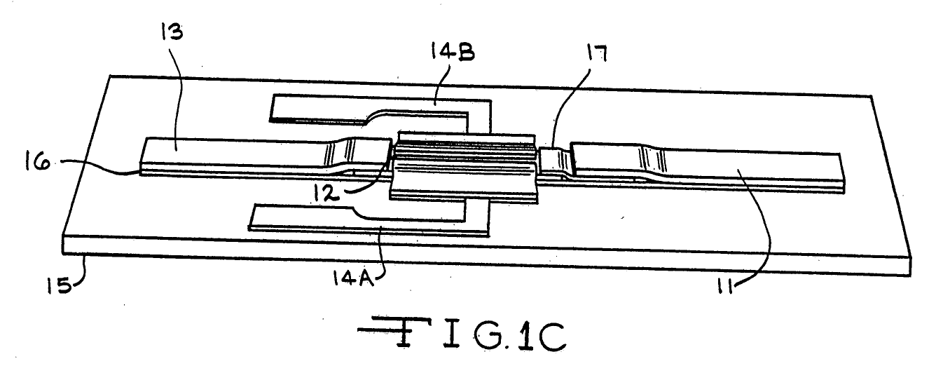

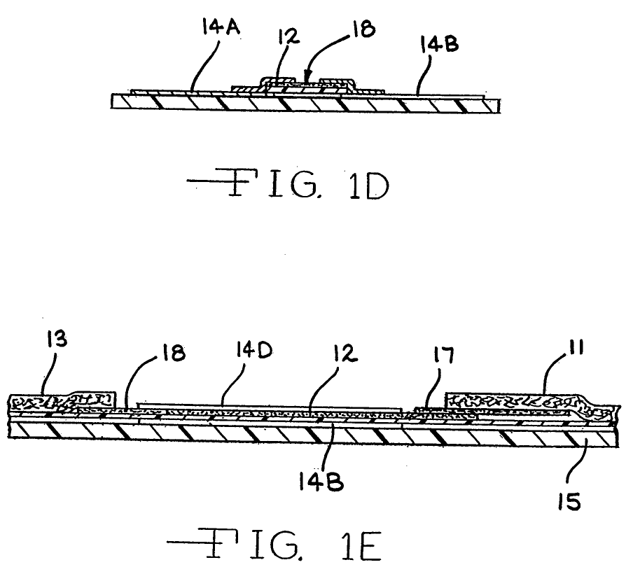

The biosensor device 10 (FIG. 1) was designed with three membranes: application membrane 11, capture or signal generation membrane 12, membrane 17 containing the conductive polymer labeled antibody and absorption membrane 13. The system was constructed as shown in FIGS. 1A, 1B and 1C with the fiber-glass (FG) membrane 11 (5×10 mm) for sample application, the nitrocellulose (NC) membrane 12 with immobilized antibody (5×20 mm) coated with silver electrodes 14C and 14D to capture the analyte, and cellulose membrane 13 (5×20 mm) for absorption of sample. The membranes 11, 12 and 13 were arranged in the order mentioned and attached onto a wafer substrate 15 using a double-sided tape 16 (FIG. 1C). Eight μl of conductive polymer (polyaniline)-labeled antibody was placed on the membrane 17 as shown in FIG. 1A.

### 1.5 Analytical Procedure

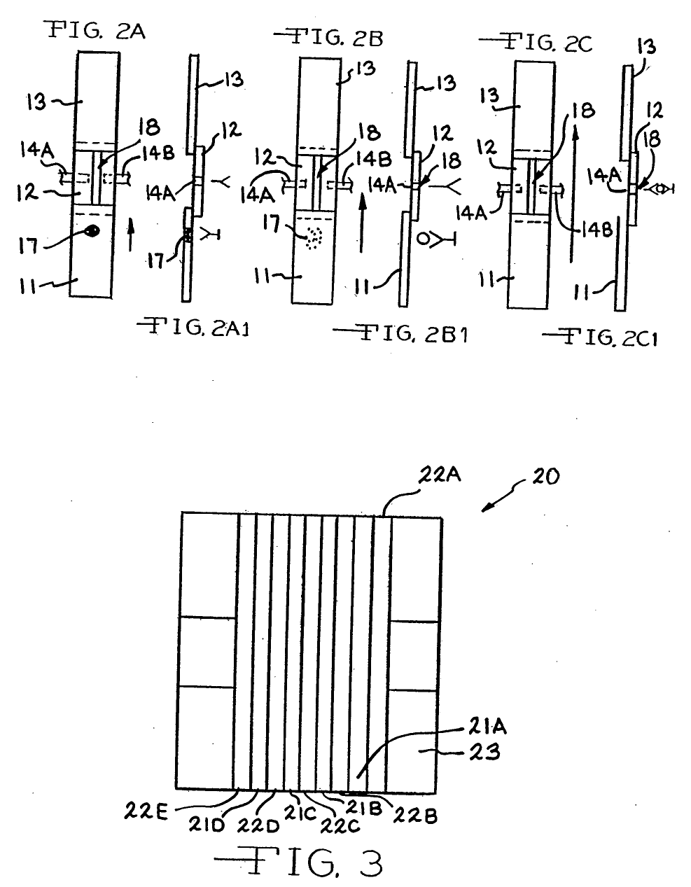

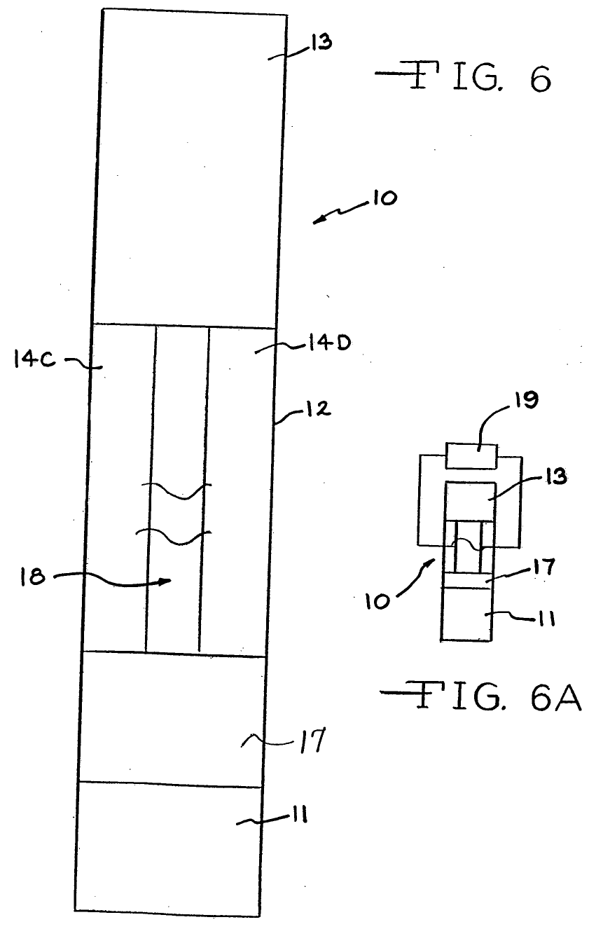

Before applying the sample onto the application membrane (or pad) 11, the resistance between the silver electrodes 14C and 14D was noted. To begin the test as shown in FIGS. 2A and 2A1, 0.1 ml of sample (containing the antigen) was dropped onto the application membrane 11. By capillary action, the solution flowed up the conjugate membrane 17 and dissolved the polyaniline-labeled antibody (FIGS. 2B and 2B1). Antibody-antigen reaction occurs and forms a complex. This complex was carried up by the migrating fluid into the capture region of membrane 12 containing the immobilized antibody (FIG. 2C, 2C1). A second antibody-antigen reaction occurred and formed a sandwich-type immune complex. The polyaniline in the sandwich complex formed a molecular wire, bridges the two silver electrodes 14C and 14D, and formed the circuit, thus generating an electrical signal. The generated signal was measured using a digital multimeter 19 (FIG. 6A) 2 to 10 minutes after the sample was applied on the application membrane 11. The concentration of the analyte was inversely proportional to the resistance across the electrodes 14C and 14D.

Without any sample, the resistance across the electrodes 14A, 14C and 14B, 14D was infinite. After sample application, the generated signal fluctuated for the first few seconds while the sample flowed by capillary action to the absorption membrane 13. Dispersion time from sample membrane 11 to absorption membrane 13 was less than one minute. When enough antigens were present, the signal stabilized and was recorded. The magnitude of signal was inversely related to antigen concentration, that is the resistance signal decreased with increasing antigen concentration. The biosensor device 10 was calibrated with the enrichment broth as the blank sample. The presence of antigen was confirmed by the standard plating method according to approved Food and Drug Administration protocol.

### Example 2

### Construction of Multi-Array

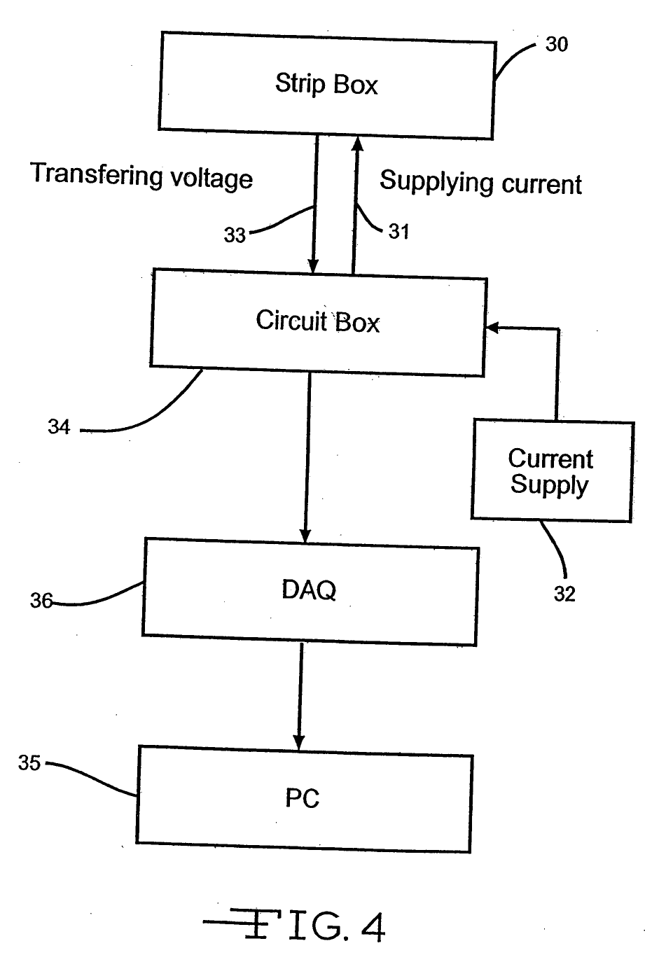

More than one capture zone is designed on the NC capture membrane 20 as shown in FIG. 3, thus, multiple types of antibodies with different specificity can be immobilized on the membrane 20. FIG. 3 shows the design of the capture or signal region 23 with multiple regions 21A to 21D for antibody immobilization between electrodes 22A to 22E. As shown in FIG. 4, after sample application, the biosensor device 10 is inserted into the strip box 30. The lead wire 31 attached to the side of the box 30 induces a constant current from the power supply 32. The current flows across the capture or signal region 23 and generates a voltage signal 33, which is proportional to the changes of the resistance. The generated voltages are transferred through a circuit box 34 and stored in the computer 35 via the data acquisition 36.

Thus the present invention:

1. Reduces the distance between electrodes to preferably less than 500 μm in order to increase desired sensitivity to 1-10 cfu.

2. Improves membrane materials to reduce detection time to less than 2 min.

3. Constructs a multi-array system for multiple and simultaneous detection.

### Example 3

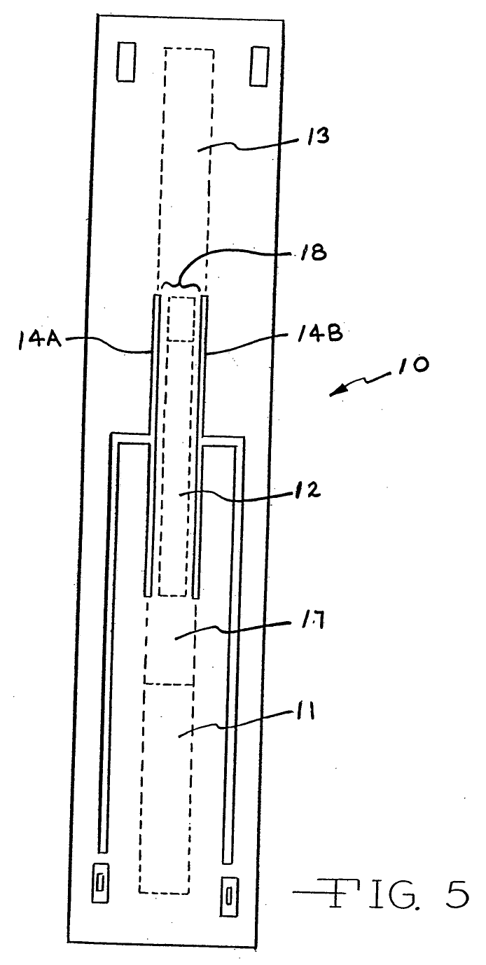

The base construction of biosensor device 10 is shown in FIG. 5. The gap 18 was 5 mm inside×30 mm long. The polyaniline membrane 17 (dotted lines) with the conjugated antibody was 10 mm long×5 mm wide in the gap 18. The sample membrane 11 was fiber glass and measured 5 mm wide and 10 mm long.

### Example 4

The device 10 was prepared as follows:

### Procedure:

### 1. Construction of the Analytical Systems

1. Prepared the model as shown in FIG. 1B above using nitrocellulose (NC) membrane 12 from Millipore (Massachusetts).

### 2. Immobilized the Antibody

1. The membrane 12 was washed 3 times with distilled water, then treated with 10% (v/v) methanol for 30 minutes and left to dry.

### PREP:

Nitrocellulose (NC) membranes were cut in to the smaller pieces (6-7 cm) to fit a petri dish. 10 ml methanol is dissolved in 100 ml of distilled water.

2. The surfaces were modified by immersing them in 0.5% (v/v) glutaraldehyde sol for 1 hour and wash with distilled water.

### PREPARATION:

0.5 ml glutaraldehyde was mixed with 100 ml distilled water. Approximately 1 ml of the solution was applied on each piece and let it be absorbed on the entire surface.

3. 0.5 mg/ml antibody was diluted into 0.02 mol phosphate buffer (PB) at pH 7.4, applied on the membrane 12, and incubated for 1 hr in the sealed container to maintain 100% humidity to immobilize the antibody.

### PREPARATION:

1. Made 0.02 mol phosphate buffer (PB)

0.02×formula weight(FW)(dibasic)×80%(of total Volume required)

0.02×FW (monobasic)×20% (of total Volume required)

Desired pH was adjusted with 1 N NaOH

After applying the antibody, the petri dish was sealed with parafilm to ensure 100% humidity.

4. The membrane was incubated in 100 mM tris buffer, pH 7.6 containing 0.1% (v/v) Tween-20 for 45 minutes and then dried off.

### PREPARATION:

Weighed 12.1 g of tris in 1 liter distilled water.

### Adjusted the pH to 7.6

### Autoclaved for 45 min

### Added 1 ml of Tween-20

### 3. Conjugating Antibody and Polyaniline

### Polyaniline Polymerization

1. 5 ml of 0.4 M of Polyaniline was mixed with 40 ml of 0.2 mM ammonium persulfate (APS), and diluted into 80 mL of 1M HCl, and reacted for 30 mins.

### PREPARATION:

Mixed 0.188 ml of aniline in 5 ml distilled water in a beaker
Added 8 ml of HCL in 80 ml distilled water in another beaker
Added 4.564 gm of APS in 100 ml distilled water

2. Wash with 5% ammonia hydroxide to rinse out excess HCl

### PREP

The initial construction was 30% by volume
Need 5%, therefore do 1:6 dilution,
Mix 10 ml ammonia hydroxide and 50 ml water

3. Filtered the product from the filter paper

4. Product was dissolved in 5 ml dimethyl formamide (DMF).

5. Undissolved product was eliminated by filtration.

6. The soluble polymer was precipitated by adding 2 mL 2M HCl in DMF

### PREPARATION:

2 ml of HCl in 10 ml distilled water

7. After filtration, residual solvent was evaporated.

8. The final protonated product was dissolved in phosphate buffer saline (PBS) containing 10% (v/v) DMF.

### Antibody Preparation:

1. Antibody was used as supplied

2. Conjugation steps

Added 800 μl of 150 μg/ml antibody in 8 ml of 0.1 g/ml polyaniline (PAN)

Let it to react for 30 mins.

Added 1 ml of 0.1 Tris 0.5% casein, left to react again for 30 mins

Spin at 13000 rpm for 3 min

Added 0.1 Tris 0.5% casein again and spin for another 3 min

The above procedure was carried out for 3 times

Lastly added 0.1 LiCl

Stored in the refrigerator when not in use

### Preparing the Conjugation Pad

The conjugation, membrane 17 was soaked with the conductive polymer labeled antibody and left to air-dry.

### Example 4

The objective of this Example was to test a spiked sample of water, apple cider and milk using the device of Example 1.

### 1. Sample Preparation

24 hours culturing of E. coli O157:H7 (ATCC #35150) (˜10⁸ cfu/ml)

The pure culture E. coli sample was diluted using serial dilution

### PREPARATION:

10 test tubes each with 9 ml of dilution blank (0.01% peptone water) (Labeled it P1 . . . P10)

Put 1 ml of the pure culture in P1 and then vortex in tubes.

Take 1 ml from test tube labeled P1 and put it in the P2 labeled test tube. Vortex in tubes.

The above procedure was repeated 10 times.

Fill ten 9 ml of the sample (water, milk or cider) in the test tube with S1 . . . S10 labels.

Take 1 ml of P1 and filled it in the S1. Vortex the test tube. Do the same for the rest of the test tubes.

### 2. Testing

1. Take 0.1 ml of S1 . . . S2 and drop it on the application pad.

2. Measure the resistance using the multimeter

3. Plating to confirm tests.

1. Take 0.1 of sample S1 . . . S2 and put it in the CHROM AGAR for E. coli confirmation.
2. Perform colony count

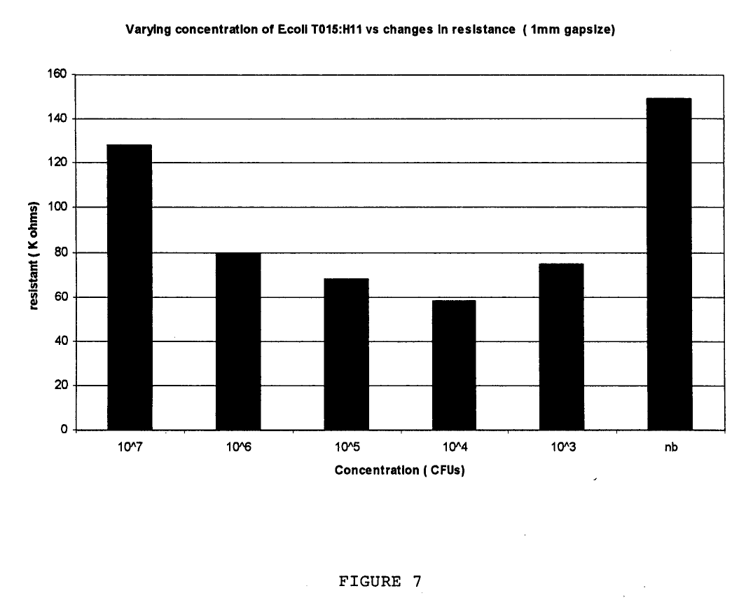

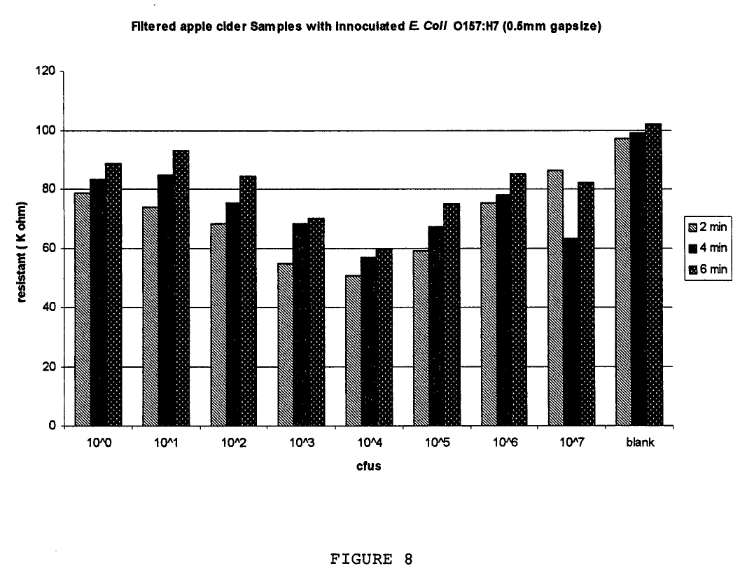

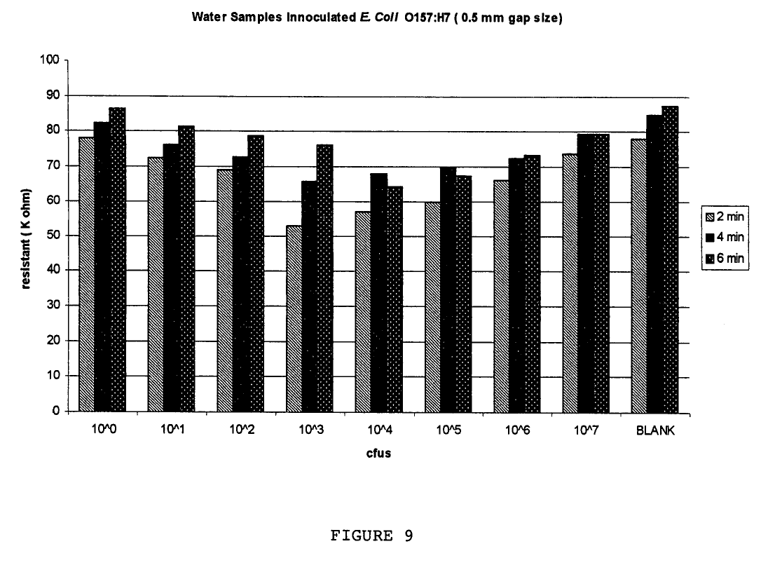

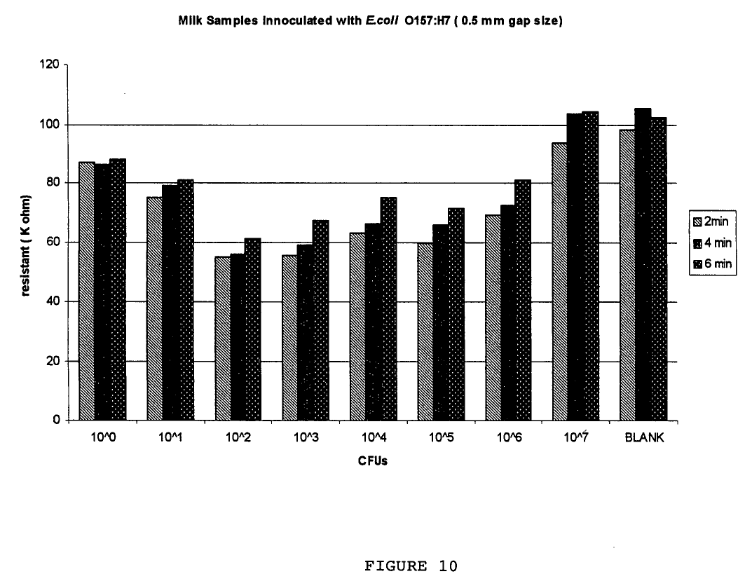

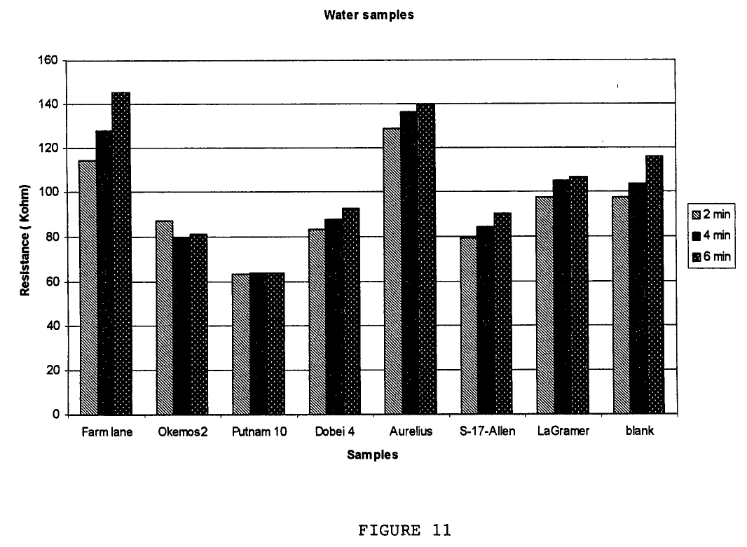

The results of testing using the biosensor device 10 are shown in FIGS. 7 to 11 for E. coli TO15:H11. It is apparent that the concentration of the specific bacteria can be determined with the device of the present invention. The bars of lower resistance in relation to the blank in FIG. 11 indicate higher levels of the E. coli.

It is intended that the foregoing description be only illustrative of the present invention and that the present invention be limited only by the hereinafter appended claims.

1-3. (canceled)

4. A method for detecting cells of a pathogen in a fluid sample which comprises:
(a) providing a biosensor device which comprises:
a strip of a substrate having at least two zones wherein a
(1) first of the zones contains a first capture reagent which binds the pathogen bound to or as a moiety of the substrate in a defined area and spaced apart electrodes defining sides of the defined area for providing an electrical bias to the defined area; and
(2) a second of the zones containing a fluid transfer medium for supplying a fluid to the first zone, wherein the second zone comprises a second defined area containing a second capture reagent which binds the pathogen bound to or as a moiety of an electrically conductive polymer, wherein when a fluid sample containing a pathogen is directly bound by the second capture reagent to form a complex, the complex migrates to the first zone in the medium and the pathogen is bound by the first capture reagent thereby altering a conductivity or resistance of the defined area in the first zone as measured between the electrodes;
(b) applying the sample to the second defined area of the second zone so that when the pathogen binds to the first and second capture reagents in the first zone, the conductivity or resistance of the first defined area is altered due to the presence of the conductive polymer to detect the pathogen.

5. The method of claim 4 wherein the device further comprises a third zone adjacent to the first zone into which the fluid is absorbed after passing through the first defined area of the first zone.

6. The method of claims 4 or 5 wherein the first defined area has a dimension between the electrodes of 1.0 mm or less between which the conductivity is measured.

7-10. (canceled)

11. A method for detecting living cells of a pathogen in a fluid sample which comprises:
(a) providing a biosensor device which comprises:
a strip of a substrate having at least two zones wherein a
(1) first of the zones contains a first antibody which binds the pathogen bound to the substrate in a defined area and spaced apart electrodes defining sides of the defined area for providing an electrical bias to the defined area; and
(2) a second of the zones containing a fluid transfer medium for supplying a fluid to the first zone, wherein the second zone comprises a second defined area containing a second antibody which binds the pathogen bound to an electrically conductive polymer, wherein when a fluid sample containing a pathogen which is bound by the second antibody bound to the conductive polymer to form a complex, the complex migrates to the first zone in the medium and the pathogen is bound by the first antibody thereby altering a conductivity or resistance of the defined area in the first zone as measured between the electrodes;
(b) applying the sample to the second defined area of the second zone so that when the pathogen binds to the first and second antibodies and the conductivity or resistance of the first defined area is altered due to the presence of the conductive polymer to detect the pathogen.

12. The method of claim 11 wherein the device further comprises a third zone adjacent to the first zone into which the fluid is absorbed after passing through the first defined area of the first zone.

13. The method of claims 11 or 12 wherein the first defined area has a dimension between the electrodes of 1.0 mm or less between which the conductivity is measured.

14. (canceled)

15. The system of claim 13 wherein the device further comprises a third zone adjacent to the first zone into which the fluid is absorbed after passing through the first defined area of the first zone.

16. (canceled)

17. The method of claims 4 or 5 wherein a pad for applying the fluid containing the pathogen is applied prior to being introduced into the second zone.

18-19. (canceled)

20. The method of claim 11 or 12 wherein a pad for applying the fluid containing the pathogen is applied prior to being introduced into the second zone.

21. The system of claim 15 wherein a pad for applying the fluid containing the pathogen is applied prior to being introduced into the second zone.

22. (canceled)

23. The method of claim 4 or 5 wherein there is a multiple array so that multiple pathogens are detected simultaneously from the same sample.

24. (canceled)

25. The method of claim 11 or 12 wherein there is a multiple array so that multiple pathogens are detected simultaneously from the same sample.

26. (canceled)

27. The method of claim 11 wherein the second capture reagent with the electrically conductive polymer which is a polyaniline has been mixed with a LiCl solution and dried in the second of the zones.
